(19) United States
(12) Patent Application Publication
(10) Pub. No.: US 2003/0045942 A1
(43) Pub. Date: Mar. 6, 2003

(54) Regenerative bone implants

(76) Inventors: Wen-Fu T. Lai, Taipei (TW); Wen-Ping Deng, Tao-Yuan (TW); Yu-Hui Tsai, Yunghe City (TW); Wing P. Chan, Taipei (TW); Wei-Chung Yang, Taipei (TW); Wellington Pham, Toledo, OH (US)

Correspondence Address: FISH & RICHARDSON PC, 225 FRANKLIN ST, BOSTON, MA, 02110, US

(21) Appl. No.: 09/946,914

(22) Filed: Sep. 5, 2001

(51) Int. Cl. A61F 2/28; C08L 1/00

(52) U.S. Cl. 623/23.51; 623/23.58; 623/23.61; 623/901; 523/115; 623/908

(57) ABSTRACT

The present invention features a regenerative bone implant. The implant includes a biocompatible and biodegradable matrix having pores, and a biocompatible and biodegradable biopolymer disposed in the pores and covalently bonded to the matrix. Optionally, the implant can further include a bone formation promoter that is also disposed in the pores and can be covalently bonded to the matrix.

### BACKGROUND

Bone implantation is necessary when a bone fails to repair itself at a normal rate or when bone loss occurs as a result of fractures or diseases. A metal implant can be used as an internal fixation to support the fracture healing, but it has limited use due to mutagenicity and mechanical properties. See, e.g., Laftman (1980) Acta Orthop Scand 51(2): 215-22; (1989) supra 60(6): 718-22; van der List et al. (1988) Acta Orthop Scand 59(3): 328-30; and Penman et al. (1984) J Bone Joint Surg Br 66(5): 632-4. A bone implant can also be a graft, such as an autograft, an allograft, or a xenograft. Use of an autograft, a tissue transplanted from one site to another in a patient, has the advantage of avoiding immune responses. However, it requires a second surgery and therefore has a higher risk of infection. An allograft is a tissue taken from a different organism of the same species, and a xenograft from an organism of a different species. Both allografts and xenografts elicit immune responses.

### SUMMARY

The present invention features a regenerative bone implant that includes a matrix having pores (including interstices), and a biopolymer disposed in the pores and covalently bonded to the matrix. Both the matrix and the biopolymer are biocompatible and biodegradable. The implant may further include a bone formation promoter that is also disposed in the pores, and if preferred, is also covalently bonded to the matrix.

The term “matrix” herein refers to a material that can be prepared from an inorganic compound (e.g., hydroxyapatite) or from an organic polymer (e.g., polylactic acid or polyglyclic acid), and is capable of mechanical strength in lieu of the bone to be replaced. The term “biopolymer” herein refers to a protein (e.g., collagen) or a protein-containing macromolecule (e.g., proteoglycan) that can function as a scaffold for cell attachment and migration to facilitate regeneration of new bone tissues. The biopolymer is disposed in the pores of the matrix for more efficient cell migration and ingrowth. A bone formation promoter is an agent that promotes growth of bone tissues and maintenance of bone mass, e.g., osteoprotegerin.

This invention also features a method for preparing a regenerative bone implant. The method includes providing a just-described matrix having pores, providing a liquid containing a just-described biopolymer, immersing the matrix in the liquid, and thereby disposing the biopolymer in the pores. It can further include covalently binding the biopolymer to the matrix. The liquid may further contain a bone formation promoter to be deposited in the pores of the matrix. Optionally, the bone formation promoter is covalently bonded to the matrix.

Also within the scope of this invention is a method for treating a bone defect in a subject by replacing the bone defect with a regenerative bone implant described above.

Other features or advantages of the present invention will be apparent from the following detailed description of several embodiments, and also from the appending claims.

### DETAILED DESCRIPTION

This invention features a regenerative bone implant that is biocompatible and biodegradable. More specifically, the implant includes a porous matrix, a biopolymer, and optionally, a bone formation promoter. The biopolymer is deposited in the pores of the matrix and covalently bonded to the matrix. The bone formation promoter, if present, is also deposited in the pores of the matrix, and may or may not be covalently bonded to the matrix.

An example of a matrix to be used to prepare an implant of this invention is a hydroxyapatite-based matrix that includes hydroxyapatite as the major component. Hydroxyapatite, naturally occurring in, e.g., bones, enamel, or dentin, has been used for years as a bone substitute or a coating material. See, for example, Frame (1987) Int. J. Oral Maxillofacial Surgery 16: 642-55, and Parsons, et al. (1988) Annals N.Y. Academy of Sciences 523: 190-207. Hydroxyapatite can be prepared by well-known methods or purchased from commercial suppliers. It is either a pure compound of Ca₁₀(PO₄)₆(OH)₂, or a composition containing other ions, such as carbonate, fluoride, chloride, or barium. When using a hydroxyapatite-based matrix to prepare a bone implant of this invention, the matrix can be hydrothermally processed to obtain desired pore sizes, such as 150 μm to 350 μm, or 200 μm to 300 μm in diameter. In order to covalently bond a biopolymer to a hydroxyapatite-based matrix, the surfaces of the matrix, in particular the interior surfaces of the pores, are first modified with functional groups, such as amino or hydroxyl. The functional groups can be introduced by plasma deposition or chemical priming. Materials used in plasma deposition include, but are not limited to, ammonia plasma, allylamine plasma, allylalcohol plasma, and plasma of any gas containing amino, hydroxyl, or other reactive groups. Compounds used in chemical priming can be amino silanes, hydroxyl silanes, or other silanes containing amino, hydroxyl, or other reactive groups. See, e.g., Sano et al. (1993) Biomaterials 14: 817-822; and Wang and Hsiue (1993) J. Polymer Science, Part A: Polymer Chemistry 31: 2601-2607.

An example of a biopolymer to be used to prepare an implant of this invention is collagen. Collagen, e.g., type I collagen, can be isolated from human or animal tissues, such as tendon, skin, bone, or ligament. See, for example, Miller and Rhodes, (1982) Methods in Enzymology 82: 33-64. It can be purified by a method of retaining the telopeptide (e.g., U.S. Pat. No. 3,114,593), or alternatively, by a method of removing the telopeptide (e.g., U.S. Pat. No. 4,233,360). It can also be reconstituted by cross-linking using a chemical reagent (e.g., U.S. Pat. Nos. 5,876,444 and 6,177,514) or by other means (e.g., UV light). Collagen can be covalently bonded to a hydroxyapatite-based matrix. The covalent bond can be formed directly between a functional group in collagen (e.g., carboxylate) and a functional group in modified hydroxyapatite (e.g., amino), or formed indirectly through a third molecule, e.g., a cross-linker. A cross-linker is an agent that has two functional groups. One of them can form a bond with the biopolymer and the other with the matrix. Examples of cross-linkers include, but are not limited to, glutaraldehyde, tresyl chloride, and N-hydroxysuccinumide.

Osteoprotegerin is an example of a bone formation promoter, which may be deposited in the pores of the matrix described above. Osteoprotegerin is a protein of the TNF receptor superfamily. It has activities associated with bone metabolism, in particular, the activity of inhibiting bone resorption thereby increasing bone density. Simonet et al. (1997) Cell 89(2): 309-19. Rat osteoprotegerin is a 401 amino acid protein, 85% and 94% homologous to mouse and human osteoprotegerins, respectively. The term “osteoprotegerin” herein refers to a polypeptide having a full or partial amino acid sequence of the rat, mouse, or human osteoprotegerin (see, for example, U.S. Pat. No. 6,015,938) or a derivative thereof, and having the activity of inhibiting bone resorption. When an implant is used for replacing a bone defect, it is preferred that it contain a bone formation promoter in a sufficient amount (e.g., 0.02% to 0.1% by weight) to promote bone growth and inhibit bone resorption. The bone formation promoter may be attached to the matrix via covalent bonding by methods well known in the art.

A bone implant of this invention can be prepared as follows: Porous hydroxyapatite is prepared by a hydrothermal process as described in, e.g., Roy and Linnehan (1974) Nature 247: 220-222, or by a process using organic particles as described in, e.g., Liu (1996) Biomaterials 17: 1955-57; and Liu (1997) Ceramic International 23: 135. During the preparation process, porous hydroxyapatite is mold into a designed shape to obtain a hydroxyapatite-based matrix. Then, the shaped matrix is immersed in a solution containing a cross-linker, which has at least two functional groups. One of the two functional groups reacts with the matrix, and a covalent bond is formed between the cross-linker and the matrix. Another solution containing a biopolymer, and optionally, a bone formation promoter is prepared. In particular, one can mix a biopolymer-containing solution with a bone formation promoter-containing solution to obtain a homogenous solution. The matrix having the cross-linker is then immersed in the just-described solution for a sufficient period of time to form another covalent bond between the cross-linker and the biopolymer (and the bone formation promoter, if present) via the second functional group in the cross-linker. The matrix is then removed from the solution and lyophilized.

If a bone formation promoter is not included in the bone implant thus obtained, it can be attached to the matrix by immersing the bone implant in a solution containing such a promoter, followed by air-drying or freeze-drying. By either method, a bone formation promoter is deposited on both the external and internal surfaces of the porous matrix.

A regenerative bone implant thus prepared can be used by following standard surgical procedures to replace a bone defect.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein, including patents, are hereby incorporated by reference in their entirety.

Preparation of Materials

Preparation of a porous hydroxyapatite-based matrix. Hydroxyapatite powder is prepared by a wet chemical method involving the reaction: 10Ca(NO₃)₂+6(NH₄)₃PO₄+2NH₃H₂O=Ca₁₀(PO₄)₆(OH)₂+20NH₄NO₃. A porous hydroxyapatite-based matrix is prepared by the following steps: (i) preparing a slurry, which includes hydroxyapatite powder, silicon carbide, magnesia, and water; (ii) molding a network substrate (e.g., polyurethane, polyvinyl chloride, or polyethyleneglycol) into a desired shape; (iii) coating the slurry onto the network substrate; and (iv) removing extra slurry by centrifugation. If necessary, steps (i)-(iv) are repeated. The thus obtained hydroxyapatite-containing substrate is sintered at a temperature of 1200° C. and then cooled down. The temperature is increased slowly so that the network substrate is decomposed gradually and no cracks are formed. Thus, a porous hydroxyapatite-based matrix is obtained, with an average pore size of 200-350 μm. After washing, the matrix is sterilized by gamma ray irradiation (20 kGy).

Extraction and purification of type I collagen. Type I collagen is extracted and purified from tendons of New Zealand white rabbits. The tendons are dissected, sliced, and washed with several changes of cold distilled water to remove plasma proteins, and then extracted by constant stirring overnight at 4° C. with 0.5 M NaCl in 50 mM Tris-HCl, pH 7.4. The supernant is decanted and the reminder is washed with several changes of cold distilled water to remove salts and then incubated overnight at 4° C. with 0.5 M HOAc pH 2.5 to obtain an aqueous extract. A salt solution (0.9 M NaCl) is added to the extract, causing precipitation. The precipitation is collected by centrifugation at 13,000 rpm for 30 min, and dissolved in 0.05 M HOAc to form a collagen-containing solution. Another salt solution (0.02 M Na₂HPO₄) is added twice to the collagen-containing solution over a 24 to 48 hr period causing precipitation. The precipitation is collected by centrifugation, and dissolved in 50 mM HOAc to obtain another collagen-containing solution. The collagen-containing solution is dialyzed against 5 mM HOAc, and finally lyophilized.

Expression of recombinant osteoprotegerin. Constructing an expression plasmid is well known in the art. See Simonet et al. (1997) Cell 89(2): 309-19. For example, Human full-length (2.4 kb) osteoprotegerin (OPG)-Fc fusion protein is constructed by PCR amplification, and cloned into the plasmid vector pCEP4 (Invitrogen, San Diego, Calif.). The pCEP4OPG-Fc vector is then lipofected into cells, e.g. 293-EBNA-1 cells (Invitrogen, San Diego, Calif.), or Chinese Hamster Ovary cells using the manufacturer's recommended methods. The OPG-Fc fusion protein is expressed, and further purified by protein A/G-affinity chromatography.

Preparation of a Bone Implant

Collagen (type I) is purified, digested with pepsin to remove the telopeptide, and reconstituted by several steps of modifications to form a glutaraldehyde-polymer amine complex (see, e.g., U.S. Pat. No. 5,876,444). Both collagen and osteoprotegerin are gamma ray sterilized and dissolved in 5 mM HOAc and a phosphate buffer saline buffer, respectively. The collagen-containing solution and the osteoprotegerin-containing solution are gently mixed and heated to 30-40° C. to facilitate mixing, if necessary. A solution containing reconstituted collagen and osteoprotegerin is obtained, and includes 0.2-1% by weight osteoprotegerin, and 99-99.8% by weight collagen.

A porous hydroxyapatite-based matrix is prepared as described above. An amino group is introduced to the surface of the porous hydroxyapatite-based matrix by ammonia plasma. Then, the matrix is immersed in a solution containing glutaraldehyde to form a covalent bond between the amino group and glutaraldehyde. The thus obtained matrix is further immersed in the above-described solution containing reconstituted collagen and osteoprotegerin for a sufficient period of time to form another covalent bound between glutaraldehyde and the collagen and between glutaraldehyde and the osteoprotegerin. Finally, the matrix is removed from the solution and lyophilized to produce a bone implant. The pore sizes of the collagen in the bone implant are in the range of 50 μm to 200 μm.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A regenerative bone implant comprising:
a biocompatible and biodegradable matrix having pores, and a biocompatible and biodegradable biopolymer disposed in the pores and covalently bonded to the matrix.

2. The regenerative bone implant of claim 1, further comprising a bone formation promoter that is also disposed in the pores.

3. The regenerative bone implant of claim 2, wherein the bone formation promoter is covalently bonded to the biocompatible and biodegradable matrix.

4. The regenerative bone implant of claim 2, wherein the biocompatible and biodegradable matrix is a hydroxyapatite-based matrix.

5. The regenerative bone implant of claim 4, wherein the biocompatible and biodegradable biopolymer is collagen.

6. The regenerative bone implant of claim 5, wherein the bone formation promoter is osteoprotegerin.

7. The regenerative bone implant of claim 2, wherein the biocompatible and biodegradable matrix is a hydroxyapatite-based matrix.

8. The regenerative bone implant of claim 2, wherein the biocompatible and biodegradable biopolymer is collagen.

9. The regenerative bone implant of claim 1, wherein the biocompatible and biodegradable matrix is a hydroxyapatite-based matrix.

10. The regenerative bone implant of claim 9, wherein the biocompatible and biodegradable biopolymer is collagen.

11. The regenerative bone implant of claim 1, wherein the biocompatible and biodegradable biopolymer is collagen.

12. A method for preparing a regenerative bone implant, comprising:
providing a biocompatible and biodegradable matrix having pores,
providing a liquid containing a biocompatible and biodegradable biopolymer,
immersing the matrix in the liquid, and thereby disposing the biopolymer in the pores.

13. The method of claim 12, further comprising covalently bonding the biopolymer in the pores to the biocompatible and biodegradable matrix.

14. The method of claim 13, wherein the biocompatible and biodegradable matrix is a hydroxyapatite-based matrix.

15. The method of claim 14, wherein the biocompatible and biodegradable biopolymer is collagen.

16. The method of claim 13, wherein the biocompatible and biodegradable biopolymer is collagen.

17. The method of claim 12, wherein the liquid further contains a bone formation promoter to be disposed in the pores.

18. The method of claim 17, further comprising covalently bonding the biocompatible and biodegradable biopolymer in the pores to the biocompatible and biodegradable matrix.

19. The method of claim 18, further comprising covalently bonding the bone formation promoter to the biocompatible and biodegradable matrix in the pores.

20. The method of claim 18, wherein the biocompatible and biodegradable matrix is a hydroxyapatite-based matrix.

21. The method of claim 20, wherein the biocompatible and biodegradable biopolymer is collagen.

22. The method of claim 21, wherein the bone formation promoter is osteoprotegerin.

23. The method of claim 18, wherein the biocompatible and biodegradable biopolymer is collagen.

24. The method of claim 23, wherein the bone formation promoter is osteoprotegerin.

25. The method of claim 18, wherein the bone formation promoter is osteoprotegerin.

26. A method for treating a bone defect in a subject, comprising replacing the bone defect with a regenerative bone implant, wherein the implant includes a biocompatible and biodegradable matrix having pores, and a biocompatible and biodegradable biopolymer disposed in the pores and covalently bonded to the matrix.

27. The method of claim 21, wherein the regenerative bone implant further includes a bone formation promoter that is also disposed in the pores.

28. The method of claim 27, wherein the bone formation promoter is covalently bonded to the biocompatible and biodegradable matrix.

29. The method of claim 27, wherein the biocompatible and biodegradable matrix is a hydroxyapatite-based matrix.

30. The method of claim 28, wherein the biocompatible and biodegradable biopolymer is collagen.

31. The method of claim 29, wherein the bone formation promoter is osteoprotegerin.

32. The method of claim 26, wherein the biocompatible and biodegradable matrix is a hydroxyapatite-based matrix.

33. The method of claim 26, wherein the biocompatible and biodegradable biopolymer is collagen.
